(19) United States
(12) Patent Application Publication
(10) Pub. No.: US 2022/0392572 A1
(43) Pub. Date: Dec. 8, 2022

(54) SYSTEMS AND METHODS FOR CONTAMINATION DETECTION IN NEXT GENERATION SEQUENCING SAMPLES

(71) Applicant: Roche Sequencing Solutions, Inc., Pleasanton, CA (US)

(72) Inventors: Olga Kamneva, Sunnyvale, CA (US); Shuo Wang, Princeton, NJ (US)

(21) Appl. No.: 17/756,068

(22) PCT Filed: Nov. 20, 2020
(86) PCT No.: PCT/EP2020/082786

§ 371 Date: May 16, 2022

Related U.S. Application Data
(60) Provisional application No. 62/938,807, filed on Nov. 21, 2019.

(51) Int. Cl. G16B 30/00; G16B 20/20; G16B 40/20; G16H 20/10

(52) U.S. Cl. CPC G16B 30/00; G16B 20/20; G16B 40/20; G16H 20/10

(57) ABSTRACT

Here we describe a statistical approach based on beta mixture modelling to detect contamination and report contamination levels as both point estimates and confidence intervals in liquid biopsy samples. We validate our method with both in silico simulation and in vitro contamination spiked samples. Although we focus on liquid biopsy samples, the same strategy is applicable to any generic NGS application with minor modifications. For example, tissue samples from a biopsy can be used according to the systems and methods described herein.

### CROSS REFERENCE TO RELATED APPLICATIONS

None.

### INCORPORATION BY REFERENCE

All publications and patent applications mentioned in this specification are herein incorporated by reference to the same extent as if each individual publication or patent application was specifically and individually indicated to be incorporated by reference.

### FIELD

Embodiments of the invention relate generally to systems and methods for next generation sequencing, and more particularly to systems and methods for contamination detection in next generation sequencing samples.

### BACKGROUND

Next generation sequencing (NGS) has advanced to the point where it can be used in diagnostic assays in various applications, such as carrier screening, infectious diseases, and cancer detection and/or analysis. In certain applications, the sequencing target, which may be a mutation (i.e., a variant) in a cancer for example, can be present in very low amounts in the sample. When the target is present is such low amounts in the sample, the risk of calling a false positive increases.

One potential source for a false positive is through sequencing error. For example, a sequencer with a 90% raw read accuracy would be expected to generate many errors for a sequence determined through a single pass, making it difficult to distinguish an error from a real mutation. One way to reduce this sequencing error is to determine a consensus sequence by sequencing the target many times, thereby achieving a desired consensus sequence accuracy (i.e., 99%, 99.9%, or 99.99%, for example).

Another source for a false positive can be from sample contamination (i.e., sample cross-contamination). However, very few methods are described in the prior art for detecting sample to sample contamination. Therefore, it would be desirable for systems and methods to be able to detect contamination in NGS samples in order to reduce the risk of false positives.

### SUMMARY OF THE DISCLOSURE

The present invention relates generally to systems and methods for next generation sequencing, and more particularly, to systems and methods for contamination detection in next generation sequencing samples.

In some embodiments, a method for detecting contamination is provided. The method can include receiving an electronic file comprising a listing of variants from a sequenced sample from a subject; calculating a set of alternative allele frequencies for a set of variants within a frequency range; determine whether the sample is contaminated based on an analysis of the set of alternative allele frequencies; if the sample is uncontaminated, administer a drug based at least in part on the listing of variants; and if the sample is contaminated, obtain an uncontaminated sequenced sample that includes a second listing of variants and administering a drug based at least in part on the second listing of variants.

In some embodiments, the frequency range is between 0 and 0.25. In some embodiments, the frequency range is between 0 and 0.1.

In some embodiments, the analysis of the set of alternative allele frequencies includes fitting the alternative allele frequencies to a clustering model. In some embodiments, the clustering model is a mixture model. In some embodiments, the mixture model is a beta mixture model.

In some embodiments, the method further includes determining whether any of the alternative allele frequencies is an outlier, and removing any outliers from the set of alternative allele frequencies before the analysis of the alternative allele frequencies.

In some embodiments, the step of determining whether any of the alternative allele frequencies is an outlier comprises a local outlier factor calculation.

In some embodiments, the method further includes determining a level of contamination from the analysis of the alternative allele frequencies.

In some embodiments, the step of determining the level of contamination includes fitting the alternative allele frequencies to a mixture model.

In some embodiments, the method further includes determining a confidence level around the level of contamination.

In some embodiments, determining a confidence level includes bootstrapping the variants and the corresponding alternative allele frequencies.

In some embodiments, the drug is a cancer drug.

In some embodiments, the cancer drug performs better on a patient having a particular variant than on a patient without the particular variant.

In some embodiments, the sample is sequenced to a mean sequencing depth of at least 1000×. In some embodiments, the sample is sequenced to a mean sequencing depth of at least 2000×.

In some embodiments, a system for detecting contamination is provided. The system include a processor programmed to execute the steps recited in any of the methods described herein.

In some embodiments, a method for detecting contamination is provided. The method can include receiving an electronic file comprising a listing of variants from a sequenced sample from a subject; calculating a set of alternative allele frequencies for a set of variants within a frequency range; and determine whether the sample is contaminated based on an analysis of the set of alternative allele frequencies.

In some embodiments, a computer product is provided. The computer product includes a computer readable medium that stores a plurality of instructions for controlling a computer system to perform an operation of any of the methods recited above.

In some embodiments, a system is provided that includes the computer product described above; and one or more processors for executing instructions stored on the computer readable medium.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a block diagram illustrating one embodiment of a computer system configured to implement one or more aspects of the present invention.

FIGS. 2A and 2B illustrate that alternative allele frequencies in a pure, uncontaminated sample tend to concentrate around three levels: 0.0 (AA), 0.5 (Aa), 1.0 (aa).

FIGS. 3A-3C illustrate the frequency distribution in the low range of alternative allele frequency for a pure sample.

FIGS. 4A-4D illustrate the frequency distribution in the low range of alternative allele frequency for a contaminated sample.

FIGS. 5A-5C illustrate the results of the simulations for the Targeted Kit panel; FIGS. 5D-5F illustrate the results of the simulations for the Expanded Kit panel; and FIGS. 5G-5I illustrate the results of the simulations for the Surveillance Kit panel.

FIG. 6 illustrates a histogram that shows the distribution of alternative allele frequencies in the low range for a contaminated sample.

FIG. 7A shows the mean of the predicted contamination level.

FIG. 7B shows the confidence level using 1000 bootstraps.

FIG. 8A illustrates the performance from 1000 synthetic samples having a single source of contamination that are sequenced to a typical coverage depth.

FIGS. 8B-8F illustrate the effect of increasing sequencing coverage for 10,000 samples at low contamination level (less than 1% contamination).

FIG. 9A illustrates the performance of the method with five sources of contamination that are sequenced to a typical coverage depth.

FIGS. 9B-9F illustrate the effect of varying the sequencing depth on 10000 synthetic samples with five sources of contamination that total to a low contamination of less than 1%.

FIG. 10 illustrates that the predicted contamination levels correspond well to the nominated contamination level, with a Pearson correlation coefficient of 0.94.

### DETAILED DESCRIPTION

Identifying sample cross-contamination is important in all next generation sequencing (NGS) applications, especially those aiming at detecting somatic mutations or any other variations that are present in the sample at low frequency, like liquid biopsies for various applications such as cancer detection and/or analysis. In such applications, contamination could lead to false positive results for key mutations and cause harm to patients, by for example, leading to the patient being given unnecessary treatment or being prescribed non-efficacious or suboptimal drugs. However, very few methods for detecting sample to sample contamination are available in the public domain. Here we describe a statistical approach based on beta mixture modelling to detect contamination and report contamination levels as both point estimates and confidence intervals in liquid biopsy samples. We validate our method with both in silico simulation and in vitro contamination spiked samples. Although we focus on liquid biopsy samples, the same strategy is applicable to any generic NGS application with minor modifications. For example, tissue samples from a biopsy can be used according to the systems and methods described herein.

### I. Next Generation Sequencing Techniques

As indicated above, the prepared nucleic acid molecules of interest (e.g., a sequencing library) are sequenced using a sequencing assay as part of the procedure for determining sequencing reads for a plurality of microsatellite loci. Any of a number of sequencing technologies or sequencing assays can be utilized. The term “Next Generation Sequencing (NGS)” as used herein refers to sequencing methods that allow for massively parallel sequencing of clonally amplified molecules and of single nucleic acid molecules.

Non-limiting examples of sequence assays that are suitable for use with the methods disclosed herein include nanopore sequencing (US Pat. Publ. Nos. 2013/0244340, 2013/0264207, 2014/0134616, 2015/0119259 and 2015/0337366), Sanger sequencing, capillary array sequencing, thermal cycle sequencing (Sears et al., Biotechniques, 13:626-633 (1992)), solid-phase sequencing (Zimmerman et al., Methods Mol. Cell Biol., 3:39-42 (1992)), sequencing with mass spectrometry such as matrix-assisted laser desorption/ionization time-of-flight mass spectrometry (MALDI-TOF/MS; Fu et al., Nature Biotech., 16:381-384 (1998)), sequencing by hybridization (Drmanac et al., Nature Biotech., 16:54-58 (1998), and NGS methods, including but not limited to sequencing by synthesis (e.g., HiSeg™, MiSeq™, or Genome Analyzer, each available from Illumina), sequencing by ligation (e.g., SOLiD™, Life Technologies), ion semiconductor sequencing (e.g., Ion Torrent™, Life Technologies), and SMRT® sequencing (e.g., Pacific Biosciences).

Commercially available sequencing technologies include: sequencing-by-hybridization platforms from Affymetrix Inc. (Sunnyvale, Calif.), sequencing-by-synthesis platforms from Illumina/Solexa (San Diego, Calif.) and Helicos Biosciences (Cambridge, Mass.), sequencing-by-ligation platform from Applied Biosystems (Foster City, Calif.). Other sequencing technologies include, but are not limited to, the Ion Torrent technology (ThermoFisher Scientific), and nanopore sequencing (Genia Technology from Roche Sequencing Solutions, Santa Clara, Calif.); and Oxford Nanopore Technologies (Oxford, United Kingdom).

### II. Exemplary Computer System for Implementing Algorithm

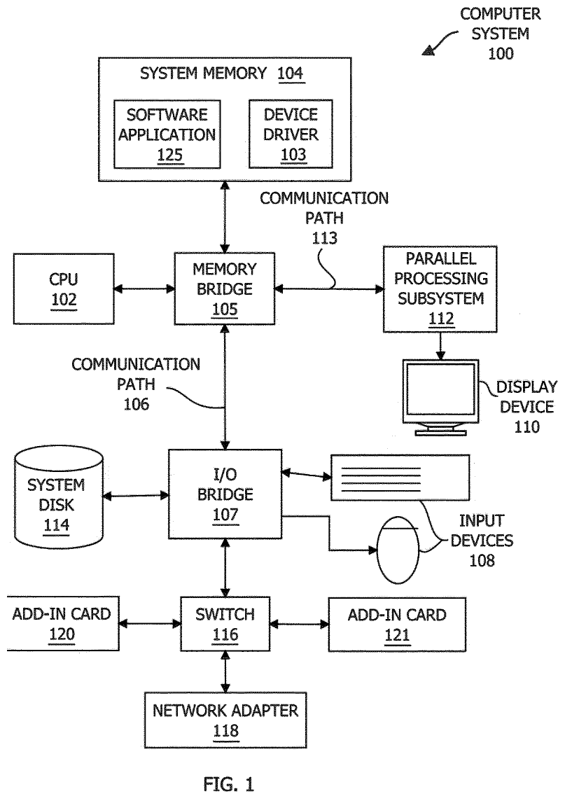

The algorithms described herein can be implemented on a computer system. For example, FIG. 1 is a block diagram illustrating one embodiment of a computer system 100 configured to implement one or more aspects of the present invention. As shown, computer system 100 includes, without limitation, a central processing unit (CPU) 102 and a system memory 104 coupled to a parallel processing subsystem 112 via a memory bridge 105 and a communication path 113. Memory bridge 105 is further coupled to an I/O (input/output) bridge 107 via a communication path 106, and I/O bridge 107 is, in turn, coupled to a switch 116.

In operation, I/O bridge 107 is configured to receive user input information from input devices 108 (e.g., a keyboard, a mouse, a video/image capture device, etc.) and forward the input information to CPU 102 for processing via communication path 106 and memory bridge 105. In some embodiments, the input information is a live feed from a camera/image capture device or video data stored on a digital storage media on which object detection operations execute. Switch 116 is configured to provide connections between I/O bridge 107 and other components of the computer system 100, such as a network adapter 118 and various add-in cards 120 and 121.

As also shown, I/O bridge 107 is coupled to a system disk 114 that may be configured to store content and applications and data for use by CPU 102 and parallel processing subsystem 112. As a general matter, system disk 114 provides non-volatile storage for applications and data and may include fixed or removable hard disk drives, flash memory devices, and CD-ROM (compact disc read-only-memory), DVD-ROM (digital versatile disc-ROM), Blu-ray, HD-DVD (high definition DVD), or other magnetic, optical, or solid state storage devices. Finally, although not explicitly shown, other components, such as universal serial bus or other port connections, compact disc drives, digital versatile disc drives, film recording devices, and the like, may be connected to I/O bridge 107 as well.

In various embodiments, memory bridge 105 may be a Northbridge chip, and I/O bridge 107 may be a Southbrige chip. In addition, communication paths 106 and 113, as well as other communication paths within computer system 100, may be implemented using any technically suitable protocols, including, without limitation, AGP (Accelerated Graphics Port), HyperTransport, or any other bus or point-to-point communication protocol known in the art.

In some embodiments, parallel processing subsystem 112 comprises a graphics subsystem that delivers pixels to a display device 110 that may be any conventional cathode ray tube, liquid crystal display, light-emitting diode display, or the like. In such embodiments, the parallel processing subsystem 112 incorporates circuitry optimized for graphics and video processing, including, for example, video output circuitry. Such circuitry may be incorporated across one or more parallel processing units (PPUs) included within parallel processing subsystem 112. In other embodiments, the parallel processing subsystem 112 incorporates circuitry optimized for general purpose and/or compute processing. Again, such circuitry may be incorporated across one or more PPUs included within parallel processing subsystem 112 that are configured to perform such general purpose and/or compute operations. In yet other embodiments, the one or more PPUs included within parallel processing subsystem 112 may be configured to perform graphics processing, general purpose processing, and compute processing operations. System memory 104 includes at least one device driver 103 configured to manage the processing operations of the one or more PPUs within parallel processing subsystem 112. The system memory 104 also includes a software application 125 that executes on the CPU 102 and may issue commands that control the operation of the PPUs.

In various embodiments, parallel processing subsystem 112 may be integrated with one or more other the other elements of FIG. 1 to form a single system. For example, parallel processing subsystem 112 may be integrated with CPU 102 and other connection circuitry on a single chip to form a system on chip (SoC).

It will be appreciated that the system shown herein is illustrative and that variations and modifications are possible. The connection topology, including the number and arrangement of bridges, the number of CPUs 102, and the number of parallel processing subsystems 112, may be modified as desired. For example, in some embodiments, system memory 104 could be connected to CPU 102 directly rather than through memory bridge 105, and other devices would communicate with system memory 104 via memory bridge 105 and CPU 102. In other alternative topologies, parallel processing subsystem 112 may be connected to I/O bridge 107 or directly to CPU 102, rather than to memory bridge 105. In still other embodiments, I/O bridge 107 and memory bridge 105 may be integrated into a single chip instead of existing as one or more discrete devices. Lastly, in certain embodiments, one or more components shown in FIG. 1 may not be present. For example, switch 116 could be eliminated, and network adapter 118 and add-in cards 120, 121 would connect directly to I/O bridge 107.

### III. Contamination Detection

In some embodiments, sample contamination can be detected by: (1) identifying a set of common single nucleotide variants (SNVs) for a particular variant based assay; (2) calculating alternative allele frequencies for the identified set of common SNVs; (3) removing outlier sites (e.g. sequencing errors and somatic mutations) with a local outlier factor (LOF); (4) fit a clustering model (i.e., a beta mixture model) on alternative allele frequencies to model background sample and foreground contamination; and (5) infer point estimate of contamination level from fitted clustering model and get confidence interval from non-parametric bootstrap.

Although the results described herein use a beta mixture model, other clustering models can be used in a similar manner. For example, connectivity models, centroid models, distribution models (i.e., mixture models), subspace models, group models, graph-based models, signed-graph models, and neural models may also be used.

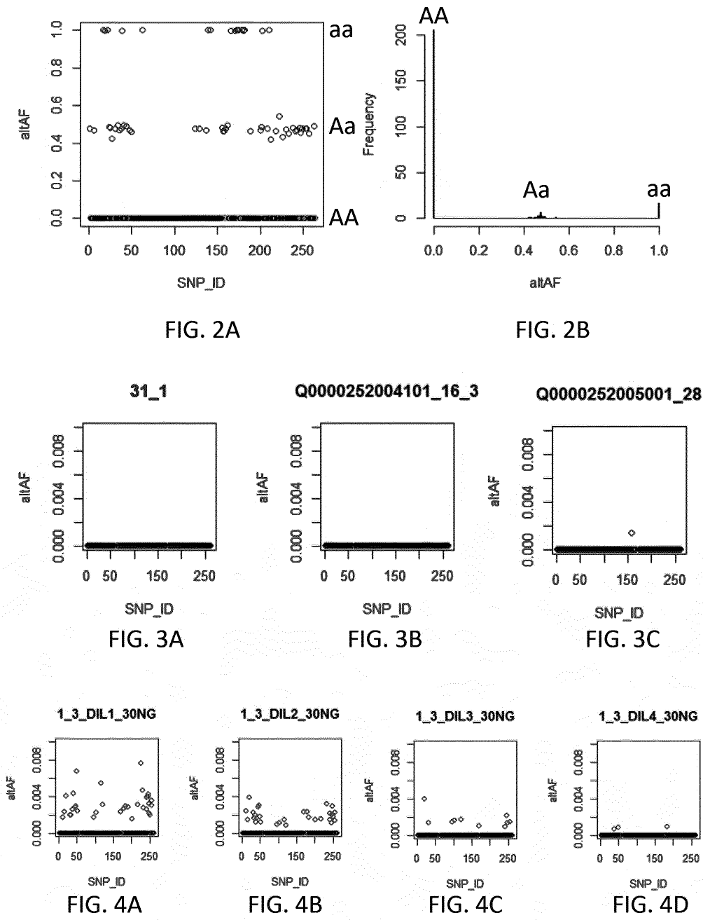

Because humans are diploid, alternative allele frequencies in a pure, uncontaminated sample tend to concentrate around three levels: 0.0 (AA), 0.5 (Aa), 1.0 (aa), as shown in FIGS. 2A and 2B. When a sample from a target subject is contaminated with sample from a non-target subject (i.e., a contaminant subject), Aa and aa SNVs from the contaminant subject shift the alternative allele frequencies of the target subject above 0 for an AA loci that has an expected level of 0.0. In short, these deviations from 0 allows us to detect contamination.

In some embodiments, the low range of alternative allele frequency (i.e., less than about 25, 20, 15, 10, or 5%) can be analyzed in order to identify pure, uncontaminated samples from contaminated samples, as shown in FIGS. 3A-3C and 4A-4D. FIGS. 3A-3C illustrate the frequency distribution in the low range of alternative allele frequency for a pure sample. As seen in FIGS. 3A-3C, very few SNVs in the low frequency range deviate from the expected 0.0 value in a pure sample. In contrast, as shown in FIGS. 4A-4D for a contaminated sample, significantly more SNVs in the low frequency range deviate from the expected 0.0 value.

In other embodiments, the high range of alternative allele frequency (i.e., greater than about 75%, 80%, 85%, 90%, or 95%) can be analyzed. In other embodiments, the middle range of alternative allele frequency (i.e., between about 25% to 75%, 30% to 70%, 35% to 65%, 40% to 60%, or 45% to 55%) can be analyzed. In other embodiments, any combination of the low range, middle range, and high range alternative allele frequency can be analyzed in order to identify pure, uncontaminated samples from contaminated samples. In other words, in some embodiments, the full frequency range from 0 to 100%, or any portion or combination of portions of the full frequency range can be used.

To build the model, we used the 1000 Genomes Project (TGP) data and identified and selected common SNVs with population alternative allele frequency over 0.5% but less than 99.5%. Consequently, we selected 285, 868, and 707 common SNVs within Targeted, Expanded and Surveillance Avenio® ctDNA Analysis Kits liquid biopsy panels. 625 relevant subjects were selected from TGP for the model, and the selection was made to reflect the US population: White (68.2%), Hispanic or Latino (15.4%), Black (11.9%), and Asian (4.5%). In other embodiments, the population selected for the model can be representative of a population in a country, state, county, province, region, continent, and the like. Factors considered for selection can include race, ethnicity, sex, age, and/or location, for example. In some embodiments, at least 100, 200, 300, 400, 500, 600, 700, 800, 900, or 1000 SNVs are selected for the model. In some embodiments, at most 100, 200, 300, 400, 500, 600, 700, 800, 900, or 1000 SNVs are selected for the model. In some embodiments, a larger panel that provides more sequencing data that covers more genes results in more SNVs that are able to be selected for the model. In some embodiments, the panel covers at least 5, 10, 15, 20, 25, 30, 35, 40, 45, 50, 55, 60, 65, 70, 75, 80, 85, 90, 95, 100, 200, 300, 400, 500, 600, 700, 800, 900, or 1000 genes. In some embodiments, the panel covers up to 5, 10, 15, 20, 25, 30, 35, 40, 45, 50, 55, 60, 65, 70, 75, 80, 85, 90, 95, 100, 200, 300, 400, 500, 600, 700, 800, 900, or 1000 genes. In some embodiments, a larger panel allows the selection criteria for the SNVs to be tightened. For example, common SNVs with population alternative allele frequency between 5 to 95%, 10 to 90%, 15 to 85%, 20 to 80%, 25 to 75%, 30 to 70%, 35 to 65%, 40 to 60%, or 45 to 55%, can be selected for the model and result in an adequate number of SNVs. In some embodiments, the population alternative frequency range can be determined based on the size of the panel.

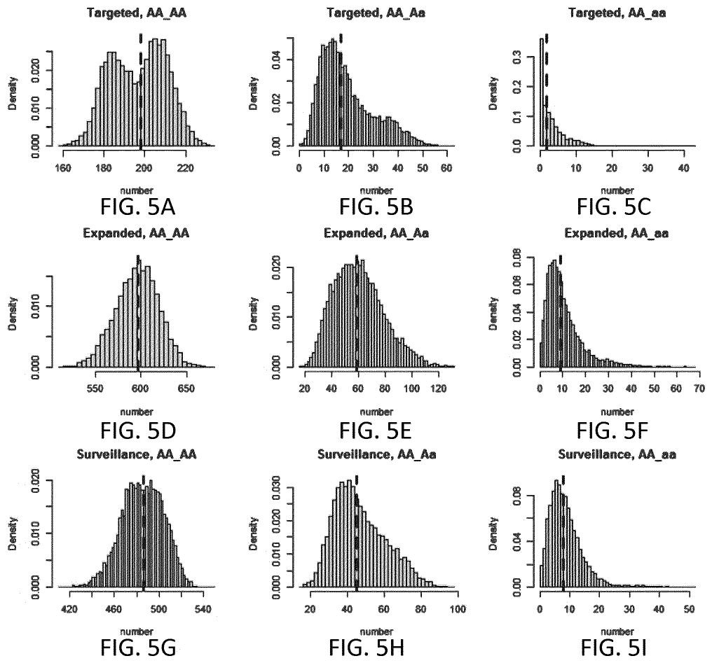

In some embodiments, the method uses the lower range of alternative allele frequency detected in liquid biopsy samples (≤25%) for modelling, informative loci. Based on 10,000 simulations for each panel, where a random target subject and a random contamination subject was selected, we show that on average we expect 21 (Targeted Kit), 70 (Expanded Kit), and 54 (Surveillance Kit) informative SNVs in samples with a single source of contamination processed using each of the three panels. FIGS. 5A-5C illustrate the results of the simulations for the Targeted Kit panel; FIGS. 5D-5F illustrate the results of the simulations for the Expanded Kit panel; and FIGS. 5G-5I illustrate the results of the simulations for the Surveillance Kit panel. In some embodiments, there are at least 5, 10, 15, 20, 25, 30, 35, 40, 45, 50, 55, 60, 65, 70, 75, 80, 85, 90, 95, or 100 informative SNVs out of the total number of SNVs selected for analysis in a sample with a single source of contamination.

We then determine alternative allele frequencies for the informative SNVs and model them using a 3-component beta mixture model for contamination detection. The general beta mixture model probability density function has the form:

f(x|π₁, π₂, α₀, β₀, α₁, β₁, α₂, β₂)=(1−π₁−π₂)Beta(x|α₀, β₀)+π₁Beta(x|α₁, β₁)+π₂Beta(x|α₂, β₂)

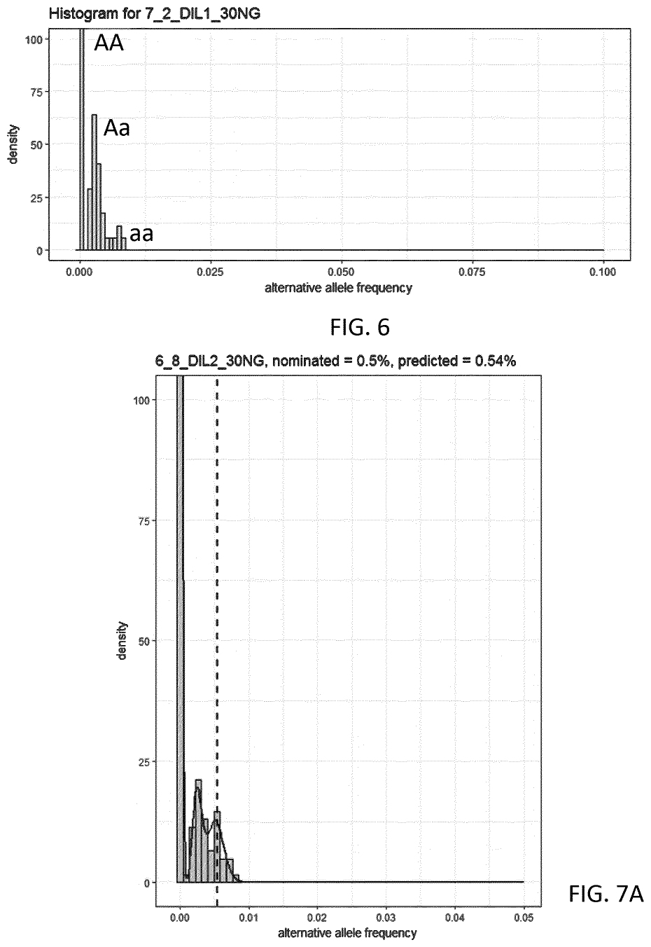

A histogram plot of the low alternative allele frequency range has only a single peak at 0.0 (not shown) for a pure sample, while a contaminated sample, assuming a single source of contamination, will have two to three peaks, including a peak at 0.0 (AA), as shown in FIG. 6. Assuming that SNVs are independent and only one source of contamination, the model will have three beta components in total: (1) background AA+contamination (AA); (2) background AA+contamination Aa; and (3) background AA+contamination aa.

We assume that each SNV is independent, each SNV is sequenced at the same depth (N, calculated as mean sequencing depth across selected sites), and there exists only one source of contamination. Therefore, the following simplifications can be used to reduce the number of parameters in the model: (1) The first beta component, shown in FIG. 6 at 0.0 alternative allele frequency (i.e., background AA with contamination AA), is directly parameterized with α₀=1, β₀=10⁴. We expect a strictly decreasing distribution (α₀=1) with a sharp spike (β₀=10⁴), as a surrogate of Dirac delta distribution, but also allows sequencing errors. (2) The second beta component corresponds to background AA and contamination Aa. The expected number of alternative alleles for each SNV belonging to this component is parameterized as α₁ and the total number of alleles per site is set to the mean coverage depth among all selected SNPs, N.Each SNV is sequenced at the same depth. Equivalently, α₁+β₁=α₂+β₂=N, where N is calculated as the mean coverage depth over all selected SNVs per sample. (3) The third beta component corresponds to background AA and contamination aa. Since aa has two alternative alleles, the expected number of alternative alleles for each SNP belonging to this component is 2α₁, with total number of alleles per site remains N. Maximum likelihood estimation is used to fit beta mixture model on alternative allele frequencies. In other words, the center of homozygous contamination distribution (aa) is twice of that for heterozygous contamination distribution (Aa), or α₂=2α₁ because of two alternative alleles.

With the above simplifications, the number of free parameters is reduced from eight to three, and the general beta mixture model is simplified to the following equation:

f(x|π₁, π₂, α₁)=(1−π₁−π₂)Beta(x|1, 10⁴)+π₁Beta(x|α₁, N−α₁)+π₂Beta(x|2α₁, N−2α₁) f(x|π₁, π₂, α₁)=(1−π₁−π₂)Beta(x|1, 10⁴)+π₁Beta(x|α₁, N−α₁)+π₂Beta(x|2α₁, N−2α₁)

A likelihood function, [symbol] (π_{1,2,}α₁|x)=Πᵢ₌₁ⁿf(xᵢ|π₁, π₂, α₁), can be used to estimate or determine parameters, π₁, π₂, α₁, of the beta mixture model with maximum likelihood, using a quasi-Newton method (i.e., limited-memory Broyden-Fletcher-Goldfarb-Shannon algorithm with bound constraints). Multiple initializations can be used to avoid local maximum.

Since maximum likelihood estimation method is sensitive to outliers (e.g. sequencing errors and somatic mutations), we use local outlier factors (LOF) to remove outliers prior to model fitting, which results in a more robust estimate of the contamination level. LOF measures the local density (lrd) of a point compared to its k nearest neighbors.

$${LOF}_{k} ( A ) = \frac{\frac{{\sum }_{B ∈ {N}_{k} ( A )} lrd ( B )}{\vert {N}_{k} ( A ) \vert}}{lrd ( A )}$$

LOF>1 means that the local density of point A is smaller than the average local density of its neighbors, indicating A is potentially an outlier. We use k=5 and a cutoff of LOF=3.

In some embodiments, other methods of outlier detection are possible. For example, in some embodiments, the outlier detection method can be univariate or multivariate. In some embodiments, the outlier detection method can be parametric or non-parametric. In some embodiments, the outlier detection method can be z-score or extreme value analysis (parametric), probabilistic and statistical modeling (parametric), linear regression models, proximity based models, information theory models, high dimensional outlier detection methods, neural networks, Bayesian networks, Hidden Markov models, fuzzy logic based methods, and/or ensemble techniques.

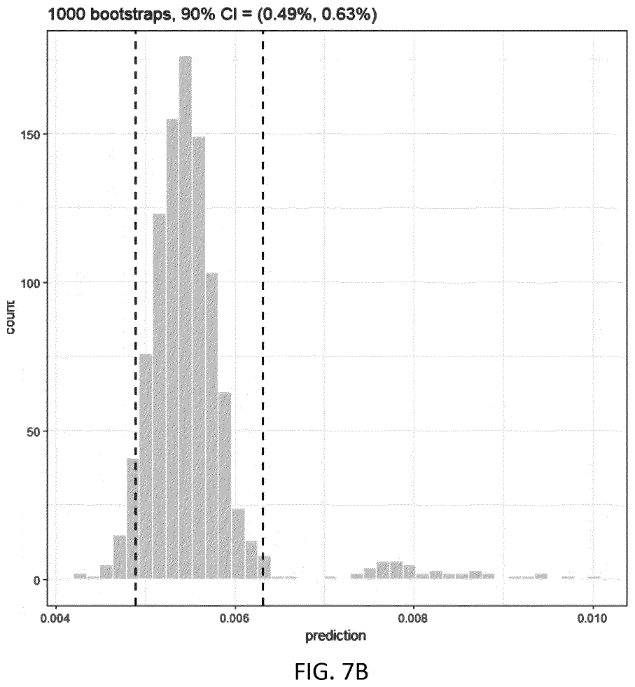

Point estimate of contamination level is then estimated as 2α₁/N (i.e. mean of the third beta component, the homozygous contamination distribution aa). FIG. 7A shows that for this example the nominated contamination level (0.5%) is in agreement with the predicted contamination level (0.54%). Confidence interval for contamination level is constructed by bootstrapping SNV sites (i.e. non-parametric bootstrap) and their corresponding alternative allele frequencies. FIG. 7B shows that with 1000 bootstraps, the 90% confidence interval of contamination level for this example is (0.49%, 0.63%). In the presence of sequencing errors, the theoretical limit of detection for our method is at least 2/N (when α₁=1, or at most one sequencing error per site). This limit gets larger when the number of sequencing errors per site gets larger (i.e. sequencing error rate gets larger). To get more conservative and less false positive results, we only report predicted contamination level larger than 4/N. In some embodiments, at least 100, 200, 300, 400, 500, 600, 700, 800, 900, 1000, 1100, 1200, 1300, 1400, 1500, 1600, 1700, 1800, 1900, 2000, 3000, 4000, 5000, 6000, 7000, 8000, 9000, or 10000 bootstraps are used to generate a confidence interval.

Comprehensive simulation study of 30,000 in silico synthetic samples from TGP demonstrates that the performance of the method increases as the sequencing depth increases, and achieves false positive rate and limit of detection of as low as 0.02% and 0.09%, respectively, for deeply sequenced samples from Expanded Avenio kit (Table 1). The target individuals and contamination individuals (one source of contamination) are again sample from the selected population from 1000 Genomes Project. We synthesize number (n) of alternative alleles for each SNV with a binomial distribution.

n˜Binom(N,p)

where N is the average sequencing depth for all sites, and p is the theoretical alternative allele frequency based on contamination level and the specific site. For p=0 or 1, we adjust it to p=2×10⁻⁵ or 1−2×10⁻⁵ to account for sequencing error. Alternative allele frequency for each site is then computed. More generally, to account for sequencing errors, p can be adjusted by an amount that corresponds to an expected magnitude of sequencing error for the system. In some embodiments, this may be the consensus sequencing error.

In some embodiments, the sequencing depth coverage is less than 25×, 50×, 75×, 100×, 200×, 300×, 400×, 500×, 600×, 700×, 800×, 900×, 1000×, 2000×, 3000×, 4000×, 5000×, 6000×, 7000×, 8000×, 9000×, or 10000×. In some embodiments, the coverage is at least 25×, 50×, 75×, 100×, 200×, 300×, 400×, 500×, 600×, 700×, 800×, 900×, 1000×, 2000×, 3000×, 4000×, 5000×, 6000×, 7000×, 8000×, 9000×, or 10000×.

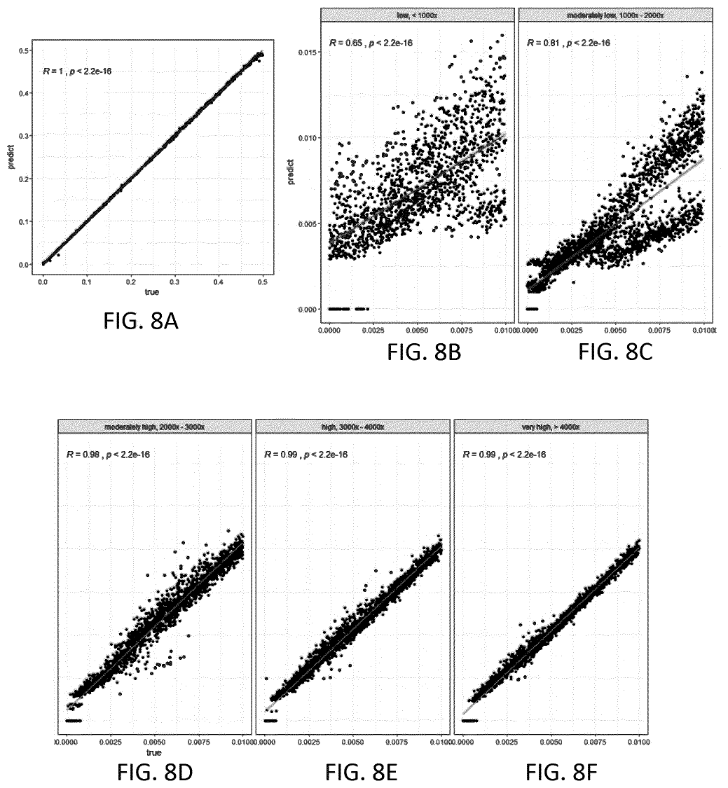

In some embodiments, the synthetic data includes samples of various contamination levels between 0 to 50% contamination. Above 50% contamination, the background contamination becomes the foreground, so there is no need to simulate a contamination level above 50%. In some embodiments, the simulated contamination level is less than 50%, 40%, 30%, 20%, 10%, 5%, 4%, 3%, 2%, or 1%. FIG. 8A illustrates the performance from 1000 synthetic samples having a single source of contamination that are sequenced to a typical coverage depth.

When the contamination level is low (i.e., less than 1%), performance begins to decay when sequencing coverage is less than about 2000×, as shown in FIGS. 8B-8F. FIGS. 8B-8F illustrate the effect of increasing sequencing coverage for 10,000 samples at low contamination level (less than 1% contamination).

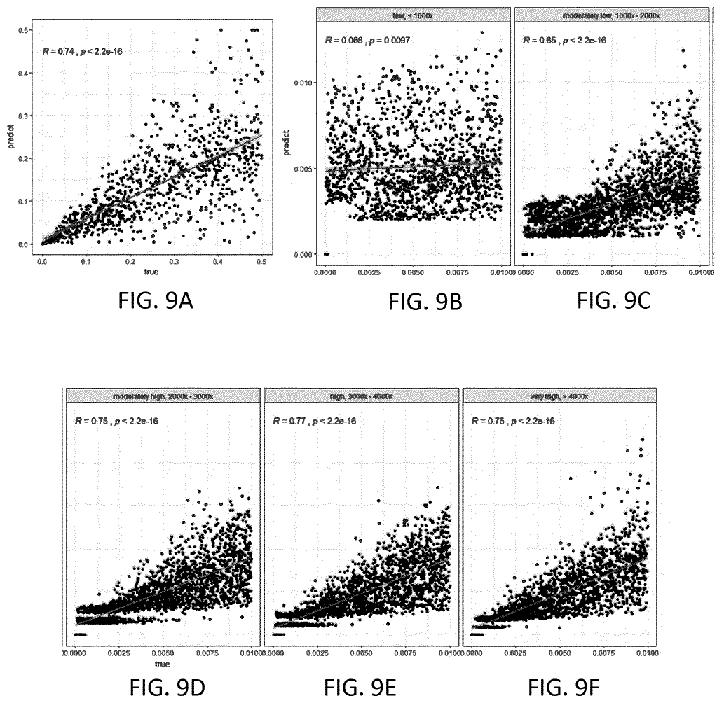

In the case of multiple sources of contamination, although one assumption of the model (i.e., one source of contamination) is violated, the method is still able to flag contaminated samples, with lower than expected predicted contamination levels. To evaluate the performance of the method for multiple sources of contamination, 1000 synthetic sample data with five sources of contamination was generated, with the contamination level from each source randomized and the total contamination level from the five sources totaling 0 to 50%. FIG. 9A illustrates the performance of the method with five sources of contamination that are sequenced to a typical coverage depth. FIGS. 9B-9F illustrate the effect of varying the sequencing depth on 10000 synthetic samples with five sources of contamination that total to a low contamination of less than 1%. Again, performance begins to decay when sequencing coverage is less than 2000×.

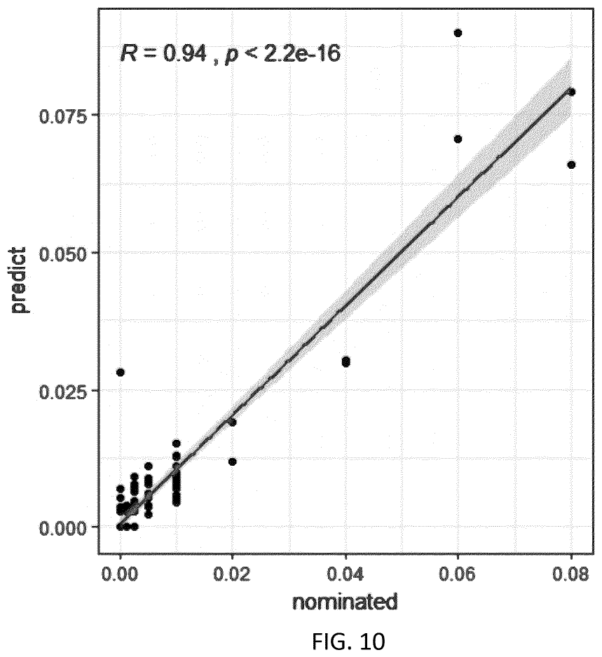

Our proposed method is further evaluated with 103 in vitro spiked plasma samples (one contamination source, 0-8% contamination level) processed using Expanded Avenio kit in CLIA lab in San Jose. The predicted contamination levels correspond well to the nominated contamination level, with a Pearson correlation coefficient of 0.94, as shown in FIG. 10.

TABLE 1
Performance of proposed method for sample contamination
on 30,000 in silico synthetic data for Expanded Avenio
kit. FPR—false positive rate; LOD—limit of detection;
CI—Clopper-Pearson exact confidence interval. LOD
determined with probit regression at 99.9% detection rate.

| | | 1 source | 5 sources |
|---|---|---|---|
| Mean coverage depth | FPR | LOD (95% CI) | LOD (95% CI) |
| <1000x | 0.135% | 0.97% | 2.12% |
| | | (0.89%-1.09%) | (1.97%-2.35%) |
| 1000x-2000x | 0.056% | 0.36% | 1.27% |
| | | (0.33%-0.40%) | (1.18%-1.40%) |
| 2000x-3000x | 0.035% | 0.12% | 0.80% |
| | | (0.11%-0.13%) | (0.74%-0.89%) |
| 3000x-4000x | 0.025% | 0.09% | 0.57% |
| | | (0.09%-0.10%) | (0.52%-0.64%) |
| >4000x | 0.020% | 0.09% | 0.41% |
| | | (0.09%-0.09%) | (0.36%-0.48%) |

The statistical methods described here can be integrated within any pipeline that produces alternative allele frequencies for multitude of sites within sequencing panels. It helps identify and allow appropriate handling of contaminated samples, which greatly increases credibility of analysis results.

### IV. Methods of Treatment

In some embodiments, the systems and methods described herein can be used to guide treatments for patients based on the correct identification of variants and not based on variants from sample contamination. For example, cancer therapies, such as the administration of cancer drugs, can be selected based on the identified variants. In some embodiments, certain treatments may be excluded because the variants have be identified as originating or potentially originating from sample contamination. In some embodiments, the patient is retested (i.e., the sample is resequenced) when sample contamination is detected, and the appropriate therapy is selected and given only after retesting and/or confirmation that the sample is not contaminated.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for detecting contamination, the method comprising:
receiving an electronic file comprising a listing of variants from a sequenced sample from a subject;
calculating a set of alternative allele frequencies for a set of variants within a frequency range;
determining whether the sample is contaminated based on an analysis of the set of alternative allele frequencies;
if the sample is uncontaminated, administering a drug based at least in part on the listing of variants; and
if the sample is contaminated, obtaining an uncontaminated sequenced sample comprising a second listing of variants and administering a drug based at least in part on the second listing of variants.

2. The method of claim 1, wherein the frequency range is between 0 and 0.25.

3. The method of claim 1, wherein the frequency range is between 0 and 0.1.

4. The method of claim 1, wherein the analysis of the set of alternative allele frequencies comprises fitting the alternative allele frequencies to a clustering model.

5. The method of claim 4, wherein the clustering model is a mixture model.

6. The method of claim 1, further comprising determining whether any of the alternative allele frequencies is an outlier, and removing any outliers from the set of alternative allele frequencies before the analysis of the alternative allele frequencies.

7. The method of claim 6, wherein the step of determining whether any of the alternative allele frequencies is an outlier comprises a local outlier factor calculation.

8. The method of claim 1, further comprising determining a level of contamination from the analysis of the alternative allele frequencies.

9. The method of claim 8, wherein the step of determining the level of contamination comprises fitting the alternative allele frequencies to a mixture model.

10. The method of claim 9, further comprising determining a confidence level around the level of contamination.

11. The method of claim 10, wherein determining a confidence level comprises bootstrapping the variants and the corresponding alternative allele frequencies.

12. The method of claim 1, wherein the drug is a cancer drug.

13. The method of claim 10, wherein the cancer drug performs better on a patient having a particular variant than on a patient without the particular variant.

14. The method of claim 1, wherein the sample is sequenced to a mean sequencing depth of at least 1000×.

15. The method of claim 1, wherein the sample is sequenced to a mean sequencing depth of at least 2000×.

16. A method for detecting contamination, the method comprising:
receiving an electronic file comprising a listing of variants from a sequenced sample from a subject;
calculating a set of alternative allele frequencies for a set of variants within a frequency range; and
determining whether the sample is contaminated based on an analysis of the set of alternative allele frequencies.

17. A computer product comprising a computer readable medium storing a plurality of instructions for controlling a computer system to perform an operation of any of the methods above.

18. A system comprising:
the computer product of claim 17; and
one or more processors for executing instructions stored on the computer readable medium.
